(19) United States
(12) Patent Application Publication
(10) Pub. No.: US 2020/0100994 A1
(43) Pub. Date: Apr. 2, 2020

(54) DEODORANT MATERIAL AND MANUFACTURING METHOD THEREOF

(71) Applicant: NAN YA PLASTICS CORPORATION, TAIPEI (TW)

(72) Inventors: TE-CHAO LIAO, TAIPEI (TW); SEN-HUANG HSU, TAIPEI (TW); WEI-SHENG CHENG, TAIPEI (TW)

(21) Appl. No.: 16/585,163

(22) Filed: Sep. 27, 2019

(30) Foreign Application Priority Data
Sep. 27, 2018 (TW) 107134161

(51) Int. Cl. A61K 8/27; A61K 8/26; A61K 8/28; A61K 8/25; A61K 8/19; A61Q 15/00; A61K 8/02

(52) U.S. Cl. CPC A61K 8/27; A61K 8/26; A61K 8/28; A61K 8/0241; A61K 8/19; A61Q 15/00; A61K 8/25

(57) ABSTRACT

A deodorant material and a manufacturing method thereof are provided. The deodorant material is a composite metal zinc silicate co-doped with a cross-group element in the composition, represented by MₓN_{y}Zn₂SiO₄, and has a multiplying and deodorizing effect on gases such as amines and acetic acid, wherein M and N are different metal elements, M is a metal element of aluminum (Al) or zirconium (Zr), N is a metal element of zirconium (Zr), tin (Sn), bismuth (Sb) or bismuth (Bi), Si is silicon, O is oxygen, x, y is a positive number, and 0<x≤1.0, 0≤y≤1.0, 0≤y/x≤1.0.

### CROSS-REFERENCE TO RELATED PATENT APPLICATION

This application claims the benefit of priority to Taiwan Patent Application No. 107134161, filed on Sep. 27, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a deodorant material and a manufacturing method thereof, and more particularly to a deodorant material in which a component is co-doped with a cross-group element to improve deodorization performance of gas such as amine or acetic acid, and has an advantage of simplifying processing steps and reducing a manufacturing cost.

### BACKGROUND OF THE DISCLOSURE

In the related art, natural plants are widely used as deodorants. For example, China Patent Publication No. CN1121834 discloses a deodorant, using natural plants as raw materials, and taking alcohol (concentration: 90-95%): orange peel: green bamboo leaves by weight ratio of (2.5 to 7.5): (0.5 to 1.5): (0.1 to 0.3). A mixture is mixed in proportion and immersed in a sealed bottle to prepare a liquid deodorant. When using, the deodorant in a bottle need only be sprayed at a location in a public place where deodorization is required, and the odor will be immediately removed. In addition to good deodorizing effect, the deodorant also has functions of sterilization and disinfection. The deodorant is not only easy to use, but also has a wide range of application, and it is non-toxic, harmless and has no side effects to the human body. However, based on the use of natural plants as raw materials, the disadvantage is that the deodorant made is not durable, have complicated processing steps and are expensive to manufacture.

For example, Chinese Patent Publication No. CN102475898A discloses an air cleaner made of pure natural plants, the main components of which are silver flower, chrysanthemum, arborvitae shell, mint leaf, benzoin, borneol, musk, sage, atractylodes, mudwort, dyer's woad, rose flower, sandalwood, fortune eupatorium herb, and dwarf sedge, and which have a certain ratio of composition. Spraying the air cleaner in the ambient environment can achieve the purpose of air purification.

In the related art, in addition to the use of natural plants as deodorants, deodorants made by mixing metal oxides with natural plants are also widely used. For example, Chinese Patent Publication No. CN102888155A discloses an odorless environment-friendly humidifying antibacterial paint, which includes 12 to 40% of emulsion, 5 to 20% of titanium white, 12 to 20% of kieselguhr, 2 to 6% of attapulgite, 4 to 8% of bamboo charcoal powder, 3 to 10% of nano TiO₂/anion powder additive, 5 to 10% of light calcium carbonate, 5 to 10% of kaolin, 1 to 5% of propanediol, 2 to 5% of assistant, and 10 to 30% of deionized water. The odorless environment-friendly humidifying antibacterial paint is applied to the interior walls of classrooms, offices, hospitals, etc., and has the effects of air-freshening, deodorization, sterilization and mildew resistance.

For example, Chinese Patent Publication No. CN101378788A discloses an aldehyde gas deodorant which is prepared by using an inorganic powder and an aninoguanidine salt to suppress the emission of an aldehyde gas. However, in the manufacturing process, it is necessary to use a large mixing device to prepare and mix the inorganic powder and the aninoguanidine salt, so that the manufacturing cost is expensive.

Chinese publication No. CN1370806 discloses a nano-antibacterial plastic in which a nano-scale inorganic oxide is selected from at least two oxides selected from the group consisting of titanium, zinc, calcium, magnesium or silver, and the total amount thereof is 0.5 to 5% by weight of the plastic resin matrix. The prepared nano-antibacterial plastic itself has functions of antibacterial, antibacterial, antiseptic and anti-mildew, and can prolong the storage period of the food, and improve the strength, hardness and service life of plastic products. However, in a manufacturing process, it is necessary to use a large mixing device to mix nano-scale inorganic oxides, so that the manufacturing cost is expensive.

In addition, Chinese Patent Publication No. CN106714850A discloses a deodorant including crystalline zinc oxide that includes zinc oxide and aluminum oxide. Further, a molar ratio of zinc oxide to aluminum oxide (ZnO/Al₂O₃) is 40 to 80, and has a high deodorizing effect on sulfur-based gas and acid gas.

### SUMMARY OF THE DISCLOSURE

In response to the above-referenced technical inadequacies, the present disclosure provides a deodorant material, especially a deodorizing agent mainly composed of a composite metal zinc silicate compound, and compound that has a high deodorizing effect on gas such as amine or acetic acid. Moreover, the processing steps are simple, the processing temperature can be lowered, and the cost is low.

In one aspect, the present disclosure provides a method for preparing a composite metal zinc silicate compound, in which a heating rate of a high-temperature furnace is controlled in a process in which a composite metal zinc silicate compound precursor is placed in the high-temperature furnace for calcination heat treatment of 2-10° C. per minute. When the high-temperature furnace is heated to a temperature of 450 to 1200° C., preferably 650 to 1000° C., the high-temperature furnace is immediately controlled to maintain a fixed temperature and subjected to calcination heat treatment under the condition where hydrogen and argon are introduced, and the heat treatment time is controlled to achieve the purpose of preparing the composite metal zinc silicate compound powder. This process not only promotes the stability of the chemical composition of the composite metal zinc silicate compound, but also reduces the variability and does not produce a composite metal zinc silicate compound with a poor proportion of elements in the component to ensure that the obtained composite metal zinc silicate compound powder has excellent deodorizing properties for ammonia-based gas or sulfur-based gas.

In one aspect, the present disclosure provides a deodorant material, which is a composite metal zinc silicate compound without adding a gelling agent, and a deodorant material having a molecular formula of MₓN_{y}Zn₂SiO₄ is synthesized by a coprecipitation method; M and N are different metal elements, M is a metal element of aluminum (Al) or zirconium (Zr), N is a metal element of zirconium (Zr), tin (Sn), bismuth (Sb) or bismuth (Bi), Si is silicon, O is oxygen, x, y is a positive number, and 0<x≤1.0, 0≤y≤1.0, 0≤y/x≤1.0. In particular, when N and Zn coexist and the molar ratio of N:Al is less than or equal to 1.0, the deodorizing multiplying effect can be exerted.

In one aspect, the present disclosure provides a deodorant material, which is a composite metal zinc silicate compound which is co-doped with an appropriate proportion of aluminum (Al) and one of the other intermetallic zirconium (Zr), tin (Sn), antimony (Sb) or antimony (Bi) metal elements. The compound has long-lasting deodorizing effect, and particularly has a deodorizing effect on gases such as amine and acetic acid.

In one aspect, the present disclosure provides a method of manufacturing the composite metal zinc silicate compound, which is to introduce a certain proportion of mixed hydrogen and argon in a heat treatment processing step to avoid partial reduction of the composite zirconium silicate to zirconia, and to weaken absorption characteristics of ammonia gas and acetic acid. At the same time, hydrogen and argon can be used to prolong the service life, that is, provide a good weather resistance.

In one aspect, the present disclosure provides a deodorant material, which is a composite metal zinc silicate compound co-doped with an appropriate proportion of zirconium (Zr) and one of intermetallic aluminum (Al), tin (Sn), antimony (Sb) and antimony (Bi). When a doping ratio of a third inter-group element to zirconium (Zr) is between 0.01 and 1.0, the higher the doping ratio is, the better the antibacterial effect under a proper high-temperature furnace heat treatment. Therefore, a manufacturer can adjust the appropriate amount of aluminum, zirconium, tin, antimony and antimony according to the requirements of antibacterial, deodorizing degree and even according to cost considerations, thereby producing a tunable deodorant material.

Therefore, the deodorant material and the manufacturing method thereof have the technical features of “the deodorant material being simple in processing and low in cost,” “the deodorant material coexisting with Zn and Al and inducing excellent deodorization effect on ammonia and acetic acid,” “the deodorant material coexisting with Zn and Zr elements and inducing excellent antibacterial properties,” and “the deodorant material having stable quality and long-lasting deodorization”.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a flow chart of steps of manufacturing an aluminum-doped composite zinc silicate of the present disclosure.

FIG. 2 is an XRD spectrum of aluminum-doped composite zinc silicate micro particles of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The deodorant material of the present disclosure is a deodorant material without adding a gelling agent, which is not only stable in quality but also has long-term deodorizing effect, especially for the purpose of absorbing ammonia gas and acetic acid, and has the following four specific embodiments:

A first embodiment is a composite metal zinc silicate compound containing zinc oxide and silicon dioxide;

A second embodiment is a composite metal zinc silicate compound doped with aluminum (Al) or zirconium (Zr) elements;

A third embodiment is a composite metal zinc silicate compound co-doped with an appropriate proportion of aluminum (Al) and one of the other intermetallic zirconium (Zr), tin (Sn), antimony (Sb) or antimony (Bi) metal elements; and

A fourth embodiment is a composite metal zinc silicate compound co-doped with an appropriate proportion of zirconium (Zr) and one of the other intermetallic tin (Sn), antimony (Sb) or antimony (Bi) metal elements.

The deodorant material provided by the present disclosure, is selected from a composite metal zinc silicate compound doped with an element such as aluminum (Al) or zirconium (Zr), and a deodorant material having a molecular formula of MxNyZn2SiO4 is synthesized by a coprecipitation method; M and N are different metal elements, M is a metal element of aluminum (Al) or zirconium (Zr), N is a metal element of zirconium (Zr), tin (Sn), bismuth (Sb) or bismuth (Bi), Si is silicon, O is oxygen, x, y is a positive number, and 0<x≤1.0, 0≤y≤1.0, 0≤y/x≤1.0.

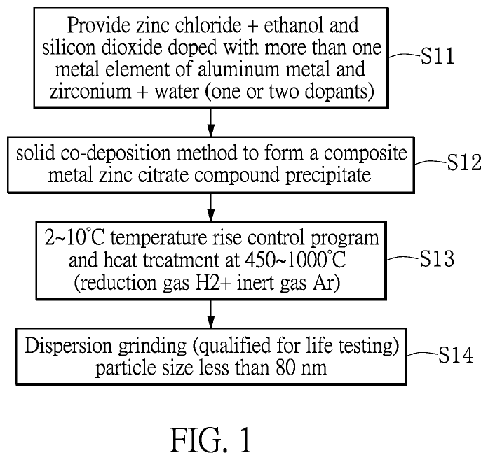

As shown in FIG. 1, processing steps of preparing the composite metal zinc silicate compound doped with aluminum (Al) or zirconium (Zr) elements are as follows:

S11: zinc chloride is dissolved ethanol to prepare a solution A, ethyl phthalate (TEOS) is dissolved in ethanol or dissolved in silicon dioxide containing aluminum metal and zirconium metal element to prepare a B solution, and use NaCl as a mineralizer and mixing chloride containing one or more metal elements of aluminum (Al), zirconium (Zr), tin (Sn), antimony (Sb) or bismuth (Bi) with water at an appropriate ratio to obtain a C solution; a preparing of the C solution is a selective step based on the component of the deodorant material.

S12: after uniformly stirring the A solution and the B solution at room temperature for 10 minutes, determine whether the C solution is added or not based on the component of the deodorant material, then heating the mixture in a 110° C. oil bath for 12 hours to obtain a composite metal zinc silicate compound precursor. The precursor is dried in an oven at 85° C. to obtain a dry powder.

S13: The dry powder is pulverized and placed in a high-temperature furnace for sintering. Before the solvent is completely evaporated at 350° C., first-stage pre-heating treatment is performed in the high-temperature furnace to raise a temperature 300 to 450° C., a heating rate is 4° C. per minute, and then at the heating rate of 2 to 10° C. per minute. When the high-temperature furnace is heated to 450 to 1200° C., preferably 650 to 1000° C., particularly preferably 800 to 1000° C., a second-stage calcination heat treatment heats for 1 to 2 hours to obtain a composite metal zinc silicate compound powder; reducing hydrogen gas and inert argon gas are introduced simultaneously with an increase of temperature and the heat treatment.

S14: grind the prepared composite metal zinc silicate compound powder to a particle size less than 3 μm, and then mix with a binder to form a slurry, and granulate by spraying to form a micron-sized composite zinc silicate micro particle powder having a particle size less than 80 nm as a main component of the deodorant material.

In the process of grinding the composite metal zinc silicate compound powder, in order to promote the uniform dispersion of the prepared composite metal zinc silicate chemical precursor particles, a specific formulation of the auxiliary agent may be added. The auxiliary agent may be selected from one or more of a coupling agent, a surfactant, a dispersing agent, a polymer modifier and a UV absorber.

X-ray diffraction identification:

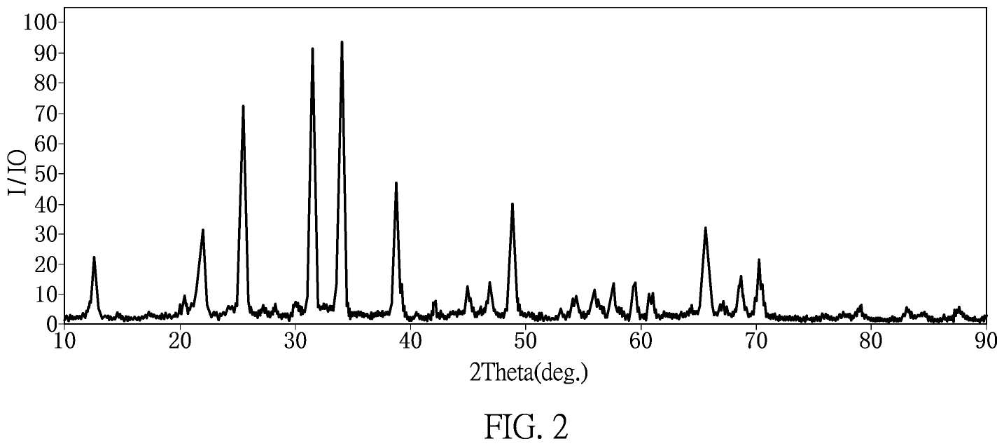

Using a Cu Ka wire of an X-ray diffractometer (manufactured by Bede Corporation, model name “D1”), measurement conditions are a tube voltage of 40 kV and a current of 40 mA, and the micron-sized composite zinc silicate fine particle powder is subjected to X-ray diffraction measurement to obtain an X-ray diffraction image as shown in FIG. 2. The X-ray diffraction image of the deodorant (d1) is shown, and a diffraction peak between 10 and 70 degrees is attributed to zinc silicate.

The following embodiments and comparative examples are given to illustrate the effects of the present disclosure, but the present disclosure is not limited thereto.

The composite metal zinc silicate compound prepared in each of the examples and the comparative examples is subjected to physical property evaluation according to the following method for absorption characteristics of ammonia gas and acetic acid:

Ammonia and acetic acid absorption characteristics test:

Ammonia and acetic acid absorption characteristics are tested using a Model 7697 overhead gas chromatography mass spectrometer (Headspace GC-Mass) manufactured by Agilent Technologies. The higher the value, the better the deodorizing effect.

### First Embodiment

A nano-sized zinc oxide solution is added to a nano-sized silicon dioxide having a solid content of 50% with respect to zinc oxide particles, stirred and uniformly mixed, and then removed by a rotary concentrator to obtain a dry powder.

The dry powder is placed in a high-temperature furnace, and a certain flow of argon and hydrogen is introduced, and the hydrogen is mainly used as a reducing gas. The process conditions are as follows: in the high-temperature furnace at a heating rate of 2° C. per minute, the high-temperature furnace is raised from room temperature to 985 to 1015° C. (average 1000° C., as indicated by the same method below), and a heat treatment is carried out for 1 hour.

A composite bismuth powder containing zinc oxide and silicon dioxide having a pH of about 7 by the above-mentioned heat treatment is added with a toluene solvent and a polymer dispersant, and is ground and dispersed by using 1 mm cerium zirconium beads to obtain a grinding dispersion containing fine particles having a particle size less than 80 nm.

A dispersion is then spray dried to remove the solvent, and liquid phase reactants are converted into small droplets via an atomizing device such as a nozzle. The small droplets pass through a high-temperature furnace tube and react rapidly to form micro particles having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Second Embodiment

Based on the principle of a coprecipitation method, a water-soluble salt is used together with a precipitant to form a poorly soluble substance, and a reaction precursor is obtained by washing and filtering, and then heated and decomposed to form a high-purity ultrafine powder. By adjusting a pH value, cations or metal ions in the solution are simultaneously precipitated due to the change of solubility to maintain uniformity of the chemical composition. This method can be carried out without special equipment and expensive raw materials. In addition, the method has the advantages of simple control, easy material acquisition, and high process reproducibility, and has the potential for mass production.

A zinc chloride is dissolved in ethanol to prepare an A solution, and TEOS is dissolved in ethanol to prepare a B solution.

The A solution and the B solution are placed in a magnetic stirrer, uniformly stirred at room temperature for 10 minutes, and then heated in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 450 to 1200° C. for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

The dispersion is then spray dried to remove the solvent, and to form micro particles having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Third Embodiment

zinc chloride is dissolved ethanol to prepare a solution A, ethyl phthalate (TEOS) is dissolved in ethanol or dissolved in silicon dioxide containing aluminum metal and zirconium metal element to prepare a B solution, and use NaCl as a mineralizer and mixing chloride containing aluminum (Al) with water at an appropriate ratio to obtain a C solution.

The A solution and the B solution are placed in a magnetic stirrer, uniformly stirred at room temperature for 10 minutes, and then heated in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 450 to 1200° C. for 1 to 2 hours, and the reducing hydrogen gas and inert argon gas are introduced simultaneously with an increase of temperature and the heat treatment.

A composite bismuth compound powder containing zinc and cerium obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

The dispersion is then spray dried to remove the solvent, and to form micro particles having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Fourth Embodiment

Firstly, zinc chloride and ethanol are mixed to obtain a transparent solution, and silicon dioxide (particle size: 3 to 4 μm) having a solid content of 100% with respect to zinc oxide particles is added. Put the two into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 450 to 1200° C. for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Fifth Embodiment

Firstly, zinc chloride and ethanol are mixed to obtain a transparent solution, and silicon dioxide (particle size: 3 to 4 μm) having a solid content of 50% with respect to zinc oxide particles is added. Put the two into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 640 to 660° C. (600° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Sixth Embodiment

Firstly, zinc chloride and ethanol are mixed to obtain a transparent solution, and silicon dioxide (particle size: 3 to 4 μm) having a solid content of 50% with respect to zinc oxide particles is added. Put the two into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 790 to 810° C. (800° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Seventh Embodiment

Firstly, zinc chloride and ethanol are mixed to obtain a transparent solution, and silicon dioxide (particle size: 3 to 4 μm) having a solid content of 20% with respect to zinc oxide particles is added. Put the two into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 790 to 810° C. (800° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours the powder are measured by injecting 30,000 ppm of ammonia gas and 30,000 ppm of acetic acid, respectively, and the results are shown in Table 1.

### Eighth Embodiment

Firstly, zinc chloride and ethanol are mixed to obtain a transparent solution, and silicon dioxide (particle size: 3 to 4 μm) having a solid content of 20% with respect to zinc oxide particles is added to obtain an A solution, and a B solution is obtained by mixing NaCl as a mineralizer and a chloride containing a zirconium (Zr) metal element with water at an appropriate ratio. Put the A solution and the B solution into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 790 to 810° C. (800° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Ninth Embodiment

Firstly, zinc chloride and ethanol are mixed to obtain a transparent solution, and a solid portion is added with respect to 20% of Al₂O₃—SiO₂ (particle size: 3 to 4 μm). Put the two into a magnetic stirrer and stir evenly at room temperature for 10 minutes, then add a coprecipitant (water or alkali compound), and then heat in a 120° C. oil bath for 6 hours to obtain a composite metal zirconate precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 640 to 660° C. (600° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Tenth Embodiment

Firstly, zinc chloride and ethanol are mixed to obtain a transparent solution, and a solid portion is added with respect to 50% of Al2O3—SiO2 (particle size: 3 to 4 μm). Put the two into a magnetic stirrer and stir evenly at room temperature for 10 minutes, then add a coprecipitant (water or alkali compound), and then heat in a 120° C. oil bath for 6 hours to obtain a composite metal zirconate precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 790 to 810° C. (800° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Eleventh Embodiment

Firstly, zinc chloride and ethanol are mixed to obtain a transparent solution, and a solid portion is added with respect to 50% of Al2O3—SiO2 (particle size: 3 to 4 μm). Put the two into a magnetic stirrer and stir evenly at room temperature for 10 minutes, then add a coprecipitant (water or alkali compound), and then heat in a 120° C. oil bath for 6 hours to obtain a composite metal zirconate precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 990 to 1010° C. (1000° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Twelfth Embodiment

Firstly, zinc chloride and ethanol are mixed to obtain a transparent solution, and a solid portion is added with respect to 50% of Al₂O₃—SiO₂ (particle size: 3 to 4 μm) to obtain a A solution, and a B solution is obtained by mixing NaCl as a mineralizer and a chloride containing a zirconium (Zr) metal element with water at an appropriate ratio. Put the A solution and the B solution into a magnetic stirrer and stir evenly at room temperature for 10 minutes, then add a coprecipitant (water or alkali compound), and then heat in a 120° C. oil bath for 6 hours to obtain a composite metal zinc silicate compound precursor. The precursor is dried in an oven at 85° C. to obtain a dry powder.

Next, the dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 640 to 660° C. (600° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm, and 0.1 g of the micro particles is placed in an overhead gas chromatography-mass spectrometry for analysis. Absorption amounts at 0 hours, 2 hours and 8 hours of the powder are measured by injecting 30,000 ppm of ammonia gas and 30,000 ppm of acetic acid, respectively, and the results are shown in Table 1.

### Comparative Example 1

A solid phase reaction method is a direct reaction of a solid phase reactant. However, it is easy to have a phenomenon of uneven mixing. Therefore, it is necessary to grind beforehand to reduce the particle size of the reactants, increase the reaction contact area and uniformity, and react at a high temperature for a long time. A synthesized product has an uneven particle size and a wide distribution range, thus after the reaction is completed, it is necessary to grind again.

Solid zinc oxide particles and silicon dioxide are ground in a mortar, and then the solid zinc oxide particles are added to the silicon dioxide having a solid content of 50% relative to the zinc oxide particles, stirred at room temperature for 30 minutes, and then uniformly mixed to obtain a composite zinc oxide and silicon dioxide precursor. Next, a dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 640 to 660° C. (650° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm. The micro particles are placed in an overhead gas chromatography-mass spectrometry for analysis, and the results are shown in Table 1.

### Comparative Example 2

Solid zinc oxide particles and silicon dioxide are ground in a mortar, and then the solid zinc oxide particles are added to the silicon dioxide having a solid content of 50% relative to the zinc oxide particles, stirred at room temperature for 30 minutes, and then uniformly mixed to obtain a composite zinc oxide and silicon dioxide precursor.

Next, a dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 790 to 810° C. (800° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm. The micro particles are placed in an overhead gas chromatography-mass spectrometry for analysis, and the results are shown in Table 1.

### Comparative Example 3

Solid zinc oxide particles and silicon dioxide are ground in a mortar, and then the solid zinc oxide particles are added to the silicon dioxide having a solid content of 50% relative to the zinc oxide particles, stirred at room temperature for 30 minutes, and then uniformly mixed to obtain a composite zinc oxide and silicon dioxide precursor.

Next, a dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 990 to 1110° C. (1000° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm. The micro particles are placed in an overhead gas chromatography-mass spectrometry for analysis, and the results are shown in Table 1.

### Comparative Example 4

Solid zinc oxide particles and the Al₂O₃—SiO₂ composite are ground in a mortar, and a solid portion is added with respect to 50% of Al₂O₃—SiO₂ (particle size: 3 to 4 μm), stirred at room temperature for 30 minutes, and then uniformly mixed to obtain a composite zinc oxide and silicon dioxide precursor.

Next, a dry powder is placed in a high-temperature furnace, and the dry powder is pulverized and sintered, and a certain flow of inert argon gas and hydrogen gas are introduced. The hydrogen gas is mainly used as a reducing gas. The processing conditions are as follows: the high-temperature furnace is raised from room temperature to 450° C. at a heating rate of 2° C. per minute, and then the heating rate is 2 to 10° C. per minute. A heat treatment is performed at a temperature of 990 to 1110° C. (1000° C.) for 1 to 2 hours, and the reducing hydrogen gas and inert argon gas are introduced simultaneously with an increase of temperature and the heat treatment.

A zinc silicate-containing composite silicide powder obtained by the above-mentioned heat treatment is added with a toluene solvent and a polymer type dispersant, and is ground and dispersed by using 1 mm cerium zirconium beads to obtain a grinding dispersion containing fine particles having a particle size less than 80 nm.

A dispersion is then spray dried to remove the solvent to form a micron-sized composite zinc silicate fine particle powder having a particle size less than 2 μm. The micro particles are placed in an overhead gas chromatography-mass spectrometry for analysis, and the results are shown in Table 1.

TABLE 1
Manufacturing methods and physical results of the embodiments
and comparative examples

| | | | | | | high- | | | | | |
|---|---|---|---|---|---|---|---|---|---|---|---|
| | | | | | | temperature | | | | | |
| | | | | | | furnace heat | | | | | |
| | | | | | | treatment | | | | | |
| | | | | | | condition | 30000 PPM | | | | |
| | | | | | | (Heating rate: | NH₃ | | | Acetic acid | |
| | | | | | Solvent removal | 2° C./min) | adsorption | | | adsorption | |
| Sample Number | | raw material | | Zn/Si | method | H₂/Ar | 0 hr | 2 hr | 8 hr | 0 hr | 2 hr |
| embodiment | 1 | Nano ZnO/H₂O | Nano SiO₂/ | 2:1 | coprecipitation + | 1000° C., 1 hr, | 92 | 92 | 92 | 92 | 92 |
| | | | H₂O | | filtration | H₂/Ar | | | | | |
| | 2 | ZnCl₂ + C₂H₅OH | TEOS + | 2:1 | coprecipitation + | 1000° C., 1 hr, | 90 | 94 | 94 | 90 | 90 |
| | | | C₂H₅OH | | filtration | H₂/Ar | | | | | |
| | 3 | ZnCl₂ + C₂H₅OH | TEOS + AlCl₃ + | 2:1:0.01 | coprecipitation + | 1000° C., 1 hr, | 92 | 96 | 96 | 92 | 92 |
| | | | C₂H₅OH | | filtration | H₂/Ar | | | | | |
| | 4 | ZnCl₂ + C₂H₅OH | SiO₂ + H₂O | 1:1 | filtration | 800° C., 1 hr, | 86 | 90 | 90 | 90 | 90 |
| | | | | | | H₂/Ar | | | | | |
| | 5 | ZnCl₂ + C₂H₅OH | SiO₂ + H₂O | 2:1 | filtration | 650° C., 1 hr, | 90 | 92 | 92 | 90 | 90 |
| | | | | | | H₂/Ar | | | | | |
| | 6 | ZnCl₂ + C₂H₅OH | SiO₂ + H₂O | 2:1 | filtration | 800° C., 1 hr, | 90 | 92 | 92 | 90 | 90 |
| | | | | | | H₂/Ar | | | | | |
| | 7 | ZnCl₂ + C₂H₅OH | SiO₂ + H₂O | 5:1 | filtration | 800° C., 1 hr, | 90 | 92 | 92 | 90 | 90 |
| | | | | | | H₂/Ar | | | | | |
| | 8 | ZnCl₂ + C₂H₅OH | SiO₂ + H₂O | 5:1 | filtration | 800° C., 1 hr, | 93 | 96 | 96 | 92 | 92 |
| | | ZrCl₄ + C₂H₅OH | | | | H₂/Ar | | | | | |
| | 9 | ZnCl₂ + C₂H₅OH | Al₂O₃—SiO₂ + | 2:1 | filtration | 650° C., 1 hr, | 93 | 96 | 96 | 92 | 92 |
| | | | H₂O | | | H₂/Ar | | | | | |
| | 10 | ZnCl₂ + C₂H₅OH | Al₂O₃—SiO₂ + | 2:1 | filtration | 800° C., 1 hr, | 93 | 96 | 96 | 92 | 92 |
| | | | H₂O | | | H₂/Ar | | | | | |
| | 11 | ZnCl₂ + C₂H₅OH | Al₂O₃—SiO₂ + | 2:1 | filtration | 1000° C., 1 hr, | 93 | 96 | 96 | 92 | 92 |
| | | | H₂O | | | H₂/Ar | | | | | |
| | 12 | ZnCl₂ + C₂H₅OH | Al₂O₃—SiO₂ + | 2:1 | filtration | 1000° C., 1 hr, | 94 | 96 | 96 | 92 | 92 |
| | | ZrCl₄ + C₂H₅OH | H₂O | | | H₂/Ar | | | | | |
| Comparative | 1 | ZnO | SiO₂ | 1:1 | No | 650° C. 1 hr, | 61 | 67 | 67 | 70 | 70 |
| example | | | | | | H₂/Ar | | | | | |
| | 2 | ZnO | SiO₂ | 1:1 | No | 800° C. 1 hr, | 62 | 66 | 66 | 71 | 71 |
| | | | | | | H₂/Ar | | | | | |
| | 3 | ZnO | SiO₂ | 2:1 | No | 800° C. 1 hr, | 64 | 68 | 68 | 72 | 72 |
| | | | | | | H₂/Ar | | | | | |
| | 4 | ZnO | Al₂O₃—SiO₂ | 2:1 | No | 1000° C. 1 hr, | 75 | 80 | 80 | 77 | 77 |
| | | | | | | H₂/Ar | | | | | |

According to Table 1, in embodiments 1 to 10, a composite metal zinc silicate compound doped with aluminum or zinc is prepared by a coprecipitation method, and the fine particles obtained after grinding, dispersing and drying are measured to confirm that the material has excellent absorption characteristics for ammonia gas. Aluminum-doped composite zinc silicate micro particles of the present disclosure do not contain a gelling agent in the process. A zinc silicate (Zn₂SiO₄) compound doped with metal ion Al is prepared by coprecipitation method and sintering reaction, which has the advantages of accurate stoichiometric ratio, high purity, good homogeneity, and reducing process temperature. Therefore, the present disclosure uses a co-precipitation process to produce aluminum-doped composite zinc silicate microparticles, which has a simple manufacturing process, can reduce the process temperature, and is low in cost.

From the results of the total index of ammonia absorption in embodiments 3, 8, 9, 10, and 11 of Table 1, it is known that a deodorant material made of aluminum (Al) or zirconium (Zr) metal has a higher ammonia absorption than the deodorant material prepared in Comparative Example 4 (a sample of zinc oxide and aluminum oxide), and shows that a material containing zinc metal silicate doped aluminum (Al) or zirconium (Zr) metal elements has excellent ammonia deodorization effect.

The Zn/Si metal doping ratios of embodiments 4, 6, and 7 are Zn:Si=1:1, 2:1, and 5:1, respectively, and the absorption of ammonia by embodiments 6 and 7 are significantly better than that of the embodiment 4, showing that the Zn/Si metal doping ratio is preferably 2:1.

From the results of the total index of ammonia absorption in embodiments 2 and 3, and 11 of Table 1, it is known that zinc silicate co-doped aluminum (Al) made a deodorant material with higher ammonia gas absorption than a single un-doped aluminum deodorant material. Therefore, the deodorant material made of zinc silicate doped with aluminum (Al) metal element has a better absorption amount of ammonia gas.

Embodiment 11 is zinc silicate co-doped aluminum (Al), and embodiment 12 is zinc silicate co-doped aluminum (Al) and zirconium (Zr). From the results of the total index of ammonia absorption in embodiments 11 and 12, it is known that zinc silicate co-doped aluminum (Al) and zirconium (Zr) has better absorption of ammonia.

From the results of the total index of the ammonia absorption amounts of embodiments 9, 10 and 11 of Table 1, it is known that, absorption characteristic of ammonia does not become better as sintering temperature gets higher. However, the higher the sintering temperature is, the higher the hardness of the sintered sample is, which is not conducive to the grinding process. Therefore, the reaction temperature is preferably greater than 650° C.

In conclusion, the present disclosure produces composite metal zinc silicate micro particles in a simplified processing step, which has the advantage of lowering the process temperature due to the precise stoichiometric ratio. In particular, composite metal zinc silicate micro particles can be made into a high-purity deodorant material, and has an excellent adsorption effect and a deodorizing effect on gases such as amines and acetic acid.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A deodorant material having a function of absorbing ammonia gas and acetic acid, characterized in that:
a composite metal zinc silicate compound containing zinc silicate and doped with aluminum (Al) or zirconium (Zr) metal element is a main component.

2. A deodorant material having the function of absorbing ammonia gas and acetic acid, characterized in that:
a composite metal zinc silicate compound represented by a molecular formula of MₓN_{y}Zn₂SiO₄, wherein M and N are different metal elements, and M is an aluminum (Al) or zirconium (Zr) metal element, N is zirconium (Zr) and tin (Sn), bismuth (Sb) or bismuth (Bi), Si is silicon, O is oxygen, x, y are positive numbers, and 0<x≤1.0, 0≤y≤1.0, 0≤y/x≤1.0.

3. A method for manufacturing a deodorant material, comprising the steps of:
(a) dissolving a precursor of zinc in ethanol to prepare an A solution;
(b) dissolving ethyl phthalate (TEOS) in ethanol or in silicon dioxide containing aluminum metal and zirconium metal element to prepare a B solution;
(c) if it is necessary to prepare a C solution according to the composition of the deodorant material, using NaCl as a mineralizer and mixing chloride containing one or more metal elements of aluminum (Al), zirconium (Zr), tin (Sn), antimony (Sb) or bismuth (Bi) with water at an appropriate ratio to obtain a C solution;
(d) after uniformly stirring the A solution and the B solution at room temperature for 10 minutes, determining whether the C solution is added or not based on the composition of the deodorant material, then heating the mixture in a 110° C. oil bath for 12 hours to obtain a composite metal zinc silicate compound precursor.
(e) after drying, the composite metal zinc silicate compound precursor is placed in a high-temperature furnace for calcination, and a calcination heat treatment is carried out at a heating rate of 2 to 10° C. per minute in the high-temperature furnace at a temperature of 650 to 1000° C. to obtain a composite metal zinc silicate compound powder; and
(f) grinding the prepared composite metal zinc silicate compound powder to a particle size less than 3 μm, and then mixing with a binder to form a slurry, and granulating by spraying to form a micron-sized composite zinc silicate micro particle powder having a particle size less than 80 nm as a main component of the deodorant material.

4. The method for manufacturing the deodorant material according to claim 3, wherein the precursor of zinc of step (a) is selected from zinc chloride, zinc nitrate and zinc acetate.

5. The method for manufacturing the deodorant material according to claim 3, wherein the precursor of step (e) is subjected to the calcination heat treatment at a temperature of 800 to 1000° C. in the high-temperature furnace.

6. The method for manufacturing the deodorant material according to claim 3, wherein during the calcination heat treatment, the precursor in step (e) is placed in the high-temperature furnace, a first-stage pre-heating treatment is performed in the high-temperature furnace to raise a temperature 300 to 450° C., and then heated to 650-1000° C. for a second-stage calcination heat treatment.

7. The method for manufacturing the deodorant material according to claim 6, wherein in step (e) of performing the calcination heat treatment, the heat treatment is performed under the condition of introducing hydrogen gas and argon gas.

8. The method for manufacturing the deodorant material according to claim 7, wherein the calcination heat treatment is carried out in step (e) for a period of 1 to 2 hours.
